(19) United States
(12) Patent Application Publication
(10) Pub. No.: US 2009/0012288 A1
(43) Pub. Date: Jan. 8, 2009

(54) Process for preparation of pentostatin (R)-3-(2-Deoxy-beta-D-erythro-pentofuranosyl)-3,6,7,8-tetrahydroimidaz[4,5-d][1,3] diazepin-8-ol

(76) Inventors: Hai-Ren Zhang, Ellicott City, MD (US); Wuyi Wang, Silver Spring, MD (US)

Correspondence Address: Jerome J. Norris, Suite 305, 1901 Pennsylvania Avenue, N.W., Washington, DC, 20006, US

(21) Appl. No.: 11/822,177

(22) Filed: Jul. 3, 2007

(51) Int. Cl. C07D 473/00

(52) U.S. Cl. 540/568

(57) ABSTRACT

In a process for preparing pentostatin, the improvement wherein reduction is performed on ketone prior to deprotection, comprising:
a) reacting 3-(2-deoxy-3,5-di-O-p-toluoyl-b-D-erythro-pentofuranosyl)-6,7-dihydroimidazol [4,5-d][1,3] diazepin-8 (3H)-one with a ruthenium catalyst formed by the reaction of di-μ-chlorobis[(p-cymene) chlororuthenium (II) and N-(arylsulfonyl)-1,2-diarylethylene diamine in a solvent;
b) stopping the reaction in step a) by making the reaction medium alkaline;
c) separating the mixture from step b) into combined organic layers and washing the reaction product from the combined organic layers with water, filtering, and evaporating solvent to provide a crude product, wherein the ratio of 8R vs 8S isomeric alcohol >100;
d) purifying said crude product by chromatography;
e) deprotecting the keto nucleoside in the crude product in methanol/sodium methoxide to obtain pentostatin; and
f) purifying by recrystallizing pentostatin from methanol to remove inorganic and isomeric impurities.

### FIELD OF THE INVENTION

The present invention relates to an improved chemical method for producing pentostatin (R)-3-(2-Deoxy-β-D-erythro-pentofuranosyl)-3,6,7,8-tetrahydroimidazo [4,5-d][1,3]diazepin-8-ol that improves the product ratio by virtue of the formation of an 8-R isomer.

### BACKGROUND OF THE INVENTION

Up to the present time, the industrial production of pentostatin has been via large scale fermentation cultures of Streptomyces antibioticus NRRL 3238, based on the process of Park-Davis Pharmaceutical¹. ¹P. W. K. Woo et al. J. Heterocycl Chem. 11: 641-645, 1974. (b) HDH Showalter et al. J. Antibiot (Tokyo). December 1992; 45 (12): 1914-8 and (c) U.S. Pat. No. 5,463,035.

### THE PRIOR ART

A chemical method of preparation of pentostatin is desired, however, there are only a few chemical syntheses that have been reported due to the fact that these syntheses face multiple technological barriers^{2,3,4}. ² (a) D. C. Baker et al. J. Am. Chem. Soc. 1979, 101, 6127-8; (b) E. Chan et al. J. Org Chem. 1982, 47, 3457-3464 and (c) H D. Showalter et al. J Med Chem October 1983; 26(10): 1478-82.³ J Z Ho et al. J. Org Chem. Jan. 10, 2003; 68(1):109-14; (b) US patent 2004/0181052 A1 and TVT Truong et al. J. Org Chem. 1993, 58, 6090-6. (d) M. Carrasco et al. Org. Syn. 1991, 70, 29-34.⁴ M. Ohno et al. J. Am. Chem Soc. 1975, 4326-4327.

A chemical synthesis method developed by Park Davis [(Ref. Footnote 2(a)) has come to be the only standard method to provide an ample quantity of the compound (amongst the chemical methods) because of a low overall yield of about 1.6%. This method starts with the synthesis of a 5-7 fused heterocyclic ring system, followed by a glycosidation step. The final conversion requires the reduction of a precursor ketone. The reduction routinely gives poor selectivity, yielding ˜1:1 of 8-R isomers. Over the years, this method has been improved [Footnote 2(c)]; however, this method is still an 11-step process that suffers from a low overall yield—again, due to the lack of chemical selectivity in several key, late stage step's.

There is a need in the production of pentostatin (R)-3-(2-Deoxy-β-D-erythro-pentofuranosyl)-3,6,7,8-tetrahydroimidazo [4,5-d][1,3] diazepin-8-ol to improve methods of preparing same so that this cytotoxic agent currently used for parenteral application for treatment of chronic lymphocytic leukemia and cutaneous T-cell lymphoma will provide improved chemical yield and economies of scale.

### SUMMARY OF THE INVENTION

One object of the present invention is to provide an improved chemical process for production of pentostatin (R)-3-(2-Deoxy-β-D-erythro-pentofuranosyl)-3,6,7,8-tetrahydroimidazo [4,5-d][1,3]diazepin-8-ol by re-designing the reaction sequence to enable better chemical maneuverability.

Another object of the present invention is to provide an improved chemical process for producing pentostatin (R)-3-(2-Deoxy-β-D-erythro-pentofuranosyl)-3,6,7,8-tetrahydroimidazo [4,5-d][1,3]diazepin-8-ol by use of transition metal catalyzed asymmetric reduction⁵ to a Pentostatin precursor ketone system to generate the chiral alcohol.⁶ ⁵ A. Fujii et al. J. Am Chem Soc. 1996, 2521-2522 and (b) G. Zassinovich et al. Chem Rev. 1992, 92, 1051-1069.⁶ M. Watanabe et al. J. Org Chem. 2002, 67, 1712-1715.

A further object to the present invention is to provide a flash chromatographic method for the purification of Pentostatin derivative {3-(2-deoxy-3,5-di-O-p-toluoyl-β-D-erythro-pentofuranosyl)-3,6,7,8-tetrahydroimidazol [4,5-d][1,3] diazepin-8-ol}. In this process, without using a tedious HPLC condition, the desired Pentostatin (8-R isomer) can be achieved to >99.0%.

These and other objects of the invention will become apparent by reference to the detailed description of the preferred embodiment of the invention hereafter.

### DETAILED DESCRIPTION OF PREFERRED EMBODIMENT OF THE INVENTION

The original chemical process of preparing pentostatin required 11 steps and was disadvantaged by three late stage technological barriers; namely, glycosidation selectivity; ketone reduction selectivity; and product purification.

The invention innovation is the discovery that any major improvement in one of these late stage steps greatly improves the synthetic value of the process.

Compared to the original reduction conditions, which gave low facial selectivity and chemical yield, modification of the reduction step proves greatly beneficial and less risky, and since the reduction step is within the three late stages, the invention process has found it judicious to focus on the reduction step.

This is done by performing the reduction step on the ketone prior to deprotection. And, since the protected ketone has broader solvent/reducer compatibility, asymmetric reductions may be achieved by using metal hydrides, boranes and transition metal catalyzed reactions.

Commencing with the metal hydrides, we achieve reductions, by using non-coordinating solvents such as toluene and CH₂Cl₂. After screening of hydrides and solvents, we found that a Superhydride® in combination with THF gives some product in low chemical yield (20-30%) and poor enantiomeric selectivity (1:1). By switching to the use of BH₃ and Corey's boride reagent, we found that little improvement in chemical yield and enantiomeric ratio is achieved.

Because of this small improvement with the hydrides, it became apparent that a broader screening of reduction conditions was needed. Parameters such as metal salt additives, reducing agents (hydride and borane), solvent, stoichiometry and temperature were screened. Solvent, the main group metal salt additive and its quantity were shown to impact the chemical yield, and as high as 85% isolated chemical yield was achieved using lithium or zinc based metal additive in the proper solvents. Still, however, the reduction selectivity remained poor.

On the other hand, employing the transition metal catalyzed reduction provided a high ratio of >100:1 in favor of the pentostatin product obtained. After further optimization, a consistent chemical yield of >75% is achieved.

In view of the combined results that the use of the ruthenium catalyst system (ruthenium with chiral diarylethylenediamine ligand) is very compatible to the nucleoside substrate, achieving other enantiomers selectively by the catalyst system is apparent.

Because of the clean result of the reduction, there is unprecedented opportunity for the use of regular chromatography technology to purify the product. At this stage, since the alcohol is fully protected, it can be easily manipulated for higher purity.

Thus, this method of selective reduction and flash column chromatography is useful for significantly larger scale chemical production of the Pentostatin compound than prior methods. Further, this process eliminates the use of HPLC purification, which is not very effective for larger scale purification.

### EXAMPLE

### Catalyst Formation

### Step 1

2.5˜5 g of di-μ-chlorobis[(p-cymene)chlororuthenium (II) and 3˜6 g of optically active N-(phenylsulfonyl)-1,2-diphenylethylenediamine (Optical purity is >98%) are mixed and stirred with lower carbon alcohols. The mixture is stirred at ambient temperature with triethylamine, and the reaction mixture is heated under an atmosphere of nitrogen for 20-25 minutes. The solution is cooled to room temperature and the reaction mixture is concentrated by a rotary evaporator and further dried under vacuum.

While the preferred aryl substituent in the N-arylsulfonyl-1,2-diarylethylene diamine is phenyl, any aryl group will suffice. For example: C₁-C₁₀ aryls, alkaryls, alkenylaryls, alkynylaryl, carbonyl-aryls, carbonyl-alkaryls, carbonyl-alkenylaryls, carboxyl-aryls, carboxyl-alkaryls, carboxyl-alkenylaryls, oxy-aryls, oxy-alkaryls, oxy-alkenylaryls, amino-aryls, amino-alkaryls, amino-alkenylaryls, amido-aryls, amido-alkaryls, and amido-alkenylaryls; heterocycles, such as pyridinyl, quinolinyl, pyrrolyl, thiophenyl, furanyl, benzofuranyl, imidazolyl, primidinyl, benzothiophenyl, benzoimidazolyl

### Asymmetric Reduction

### Step 2

To a reactor is added 0.3˜0.6 g of 3-(2-deoxy-3,5-di-O-p-toluoyl-b-D-erythro-pentofuranosyl)-6,7-dihydroimidazol [4,5-d][1,3]diazepin-8 (3H)-one. To this is added the above-described catalyst (in its entirety) in 0.3˜0.6 mL of dichloromethane. The solution is diluted with additional dichloromethane. Triethylamine (0.216 mL) and formic acid (0.07 mL) is added to the reactor sequentially. The reaction is then stopped by making the medium slightly alkalinic, pH ˜8. The mixture is separated. Layers are mixed vigorously and allowed to settle. The combined organic layers are washed twice with water and dried over 1.50 g of anhydrous sodium sulfate. The product is filtered to a 5.0 mL evaporating flask. Solvent is evaporated to give a crude product with yield of alcohol being greater than 80%. The ratio of 8R vs 8S is >100.

Starting material solvent compatibility and unprecedented selectivity is achieved in this step.

### Purification by Chromatography

### Step 3

The above crude product (27 g˜33 g) is loaded to a silica gel column. The separation is carried out using a solvent system consisting of hexane, ethyl acetate and methanol. The purification process is monitored by thin layer chromatographic method. The portions containing the desired product are pooled and the solvent is evaporated to dryness. 1˜3 g of activated charcoal is added to remove color. The mixture is filtered through a bed of Celite using a Buchner funnel. The filtrate is transferred to a round bottom flask from which the solvent is evaporated via a rotary evaporator, to yield the alcohol product: ˜22 g, ˜73%.

This is an unprecedented convenience for late stage chromatography purification.

### Alcohol Product (15-25 g) from Above Step is Charged to a Reactor

### Step 4

Appropriate amount of anhydrous methanol is added. The resulting solution is cooled in an ice bath. Small amount of sodium methoxide is added. The mixture is warmed up and stirred at 20° C. The reaction mixture is re-cooled to −78° C. A slow stream of carbon dioxide gas is bubbled into the solution for 1.5˜2 hours. The resulting precipitate is collected by suction filtration.

This step of deprotection as part of the new process obtains the pentostatin product by crystallization.

### Purification By Recrystallization from Methanol to Remove Inorganic and Isomeric Impurities

### Step 5

The filtrate from the above step is transferred to a reactor, from which the solvent is evaporated. The solid residue is taken up by ether and filtered. The cake is rinsed with ether multiple times to remove organic impurites. After being air-dried for 15˜20 min, the crude product weighed 22 g. The product is transferred to a flask, followed by addition of a solvent system consisting of CH₂Cl₂ and MeOH. The suspension is stirred and then filtered. The filter cake is rinsed with the same solvent system three times. The solid is subjected to vacuum to remove the solvent. The gross weight is 3.1 g. This product can be further crystallized from low carbon alcohols.

As may be seen from the foregoing, the invention process of producing pentostatin utilizes the sequence of reduction-purification—deprotection, and this sequence is superior to the process sequence of deprotection-reduction-purification, in that the invention process obtains higher purity pentostatin and a higher quantity of pentostatin.

While not wishing to be bound by theory as to why the invention process results in higher purity and a higher yield or quantity of pentostatin, it is believed that the ruthenium catalyst's compatibility to the pentostatin nucleoside substrate achieves far greater selectivity than that which was previously known.

Further, the invention method appears additionally important in that it may be utilized to make other isomers in a selective manner.

As a result of using the invention process for making pentostatin, we are placed in a better position to use flash chromatography instead of HPLC for purification, to thereby effectively provide pentostatin >99%, and in larger quantities.

In isocratic HPLC the analyte is forced through a column of the stationary phase (usually a tube packed with small round particles with a certain surface chemistry) by pumping a liquid (mobile phase) at high pressure through the column. The sample to be analyzed is introduced in a small volume to the stream of mobile phase and is retarded by specific chemical or physical interactions with the stationary phase as it traverses the length of the column. The amount of retardation depends on the nature of the analyte, stationary phase and mobile phase composition. The time at which a specific analyte elutes (comes out of the end of the column) is called the retention time and is considered a reasonably unique identifying characteristic of a given analyte. The use of pressure increases the linear velocity (speed) giving the components less time to diffuse within the column, leading to improved resolution in the resulting chromatogram. Common solvents used include any miscible combinations of water or various organic liquids (the most common are methanol and acetonitrile). Water may contain buffers or salts to assist in the separation of the analyte components, or compounds such as Trifluoroacetic acid which acts as an ion pairing agent.

On the other hand, flash chromatography is a rapid form of preparative column chromatography based on optimised pre-packed columns through which is pumped solvent at a high flow rate. It is a simple and economical approach to Preparative LC, and utilizes a plastic column filled with some form of solid support, usually silica gel, with the sample to be separated placed on top of this support. The rest of the column is filled with an isocratic or gradient solvent which, with the help of pressure, enables the sample to run through the column and become separated. Flash chromatography may use air pressure as in the case when it was initially founded, or pumps as used presently to speed up the separation.

We claim:

1. A method for preparing a catalyst that is compatible to the pentostatin nucleoside substrate to achieve improved stereoselective reducton of the ketone functionality to provide an unusually high stereo ratio of >100 in favor of the formation of the desired 8-R isomer comprising:
a) reacting 3-(2-deoxy-3,5-di-O-p-toluoyl-b-D-erythro-pentofuranosyl)-6,7-dihydroimidazol [4,5-d][1,3]diazepin-8 (3H)-one with a ruthenium catalyst formed by the reaction of di-μ-chlorobis[(p-cymene) chlororuthenium (II) and N-(arylsulfonyl)-1,2-diarylethylene diamine in a solvent;
b) stopping the reaction in step a) by making the reaction medium alkaline;
c) separating the mixture from step b) into combined organic layers and washing the reaction product from said combined organic layers with water, filtering, and evaporating solvent to provide a crude product, wherein the ratio of 8R vs 8S isomeric alcohol >100.

2. The process of claim 1 wherein the aryl group in the N-(arylsulfonyl)-1,2-diarylethylene diamine is selected from the group consisting of C₁-C₁₀ aryls, alkaryls, alkenylaryls, alkynylaryl, carbonyl-aryls, carbonyl-alkaryls, carbonyl-alkenylaryls, carboxyl-aryls, carboxyl-alkaryls, carboxyl-alkenylaryls, oxy-aryls, oxy-alkaryls, oxy-alkenylaryls, amino-aryls, amino-alkaryls, amino-alkenylaryls, amido-aryls, amido-alkaryls, and amido-alkenylaryls; heterocycles, such as pyridinyl, quinolinyl, pyrrolyl, thiophenyl, furanyl, benzofuranyl, imidazolyl, primidinyl, benzothiophenyl, benzoimidazolyl.

3. The process of claim 2 wherein the aryl group is phenyl.

4. The process of claim 3 wherein the N-(phenylsulfonyl)-1,2-diphenylethylenediamine is optically active.

5. The process of claim 4 wherein the optically active N-(phenylsulfonyl)-1,2-diphenylethylenediamine has an optical purity >98%.

6. In a process for preparing pentostatin, the improvement wherein reduction is performed on ketone prior to deprotection, comprising:
a) reacting 3-(2-deoxy-3,5-di-O-p-toluoyl-b-D-erythro-pentofuranosyl)-6,7-dihydroimidazol [4,5-d][1,3]diazepin-8 (3H)-one with a ruthenium catalyst formed by the reaction of di-μ-chlorobis[(p-cymene) chlororuthenium (II) and N-(arylsulfonyl)-1,2-diarylethylene diamine in a solvent;
b) stopping the reaction in step a) by making the reaction medium alkaline;
c) separating the mixture from step b) into combined organic layers and washing the reaction product from said combined organic layers with water, filtering, and evaporating solvent to provide a crude product, wherein the ratio of 8R vs 8S isomeric alcohol >100;
d) purifying said crude product by chromatography;
e) deprotecting the keto nucleoside in said crude product in methanol/sodium methoxide to obtain pentostatin; and
f) purifying by recrystallizing pentostatin from methanol to remove inorganic and isomeric impurities.

7. The process of claim 6 wherein the aryl group in the N-(arylsulfonyl)-1,2-diarylethylene diamine is selected from the group consisting of C₁-C₁₀ aryls, alkaryls, alkenylaryls, alkynylaryl, carbonyl-aryls, carbonyl-alkaryls, carbonyl-alkenylaryls, carboxyl-aryls, carboxyl-alkaryls, carboxyl-alkenylaryls, oxy-aryls, oxy-alkaryls, oxy-alkenylaryls, amino-aryls, amino-alkaryls, amino-alkenylaryls, amido-aryls, amido-alkaryls, and amido-alkenylaryls; heterocycles, such as pyridinyl, quinolinyl, pyrrolyl, thiophenyl, furanyl, benzofuranyl, imidazolyl, primidinyl, benzothiophenyl, benzoimidazolyl.

8. The process of claim 7 wherein the aryl group is phenyl.

9. The process of claim 6 wherein in step d) said chromatography is flash chromatography.
